(19) United States
(12) Patent Application Publication
(10) Pub. No.: US 2022/0218404 A1
(43) Pub. Date: Jul. 14, 2022

(54) MONOPOLAR ELECTROSURGERY BLADE AND ELECTROSURGERY BLADE ASSEMBLY

(71) Applicant: I.C. Medical, Inc., Phoenix, AZ (US)

(72) Inventor: Ioan Cosmescu, Phoenix, AZ (US)

(21) Appl. No.: 17/710,689

(22) Filed: Mar. 31, 2022

Related U.S. Application Data
(63) Continuation of application No. 15/695,642, filed on Sep. 5, 2017.
(60) Provisional application No. 62/383,851, filed on Sep. 6, 2016.

(51) Int. Cl. A61B 18/14; A61L 31/02

(52) U.S. Cl. CPC A61B 18/14; A61B 18/1402; A61B 2018/00077; A61L 31/026; A61L 31/022

(57) ABSTRACT

Electrosurgery blades including electrosurgery blade assemblies having argon beam capability. The electrosurgery blade includes a non-conductive planar member having opposite planar sides with a bottom angled sharp cutting edge, and a conductive layer located on one or both of the opposing planar sides of the non-conductive planar member where the conductive layer lies adjacent to the angled sharp cutting edge of the non-conductive planar member without covering the angled sharp cutting edge. In embodiments of the electrosurgery blade assemblies having argon beam capability, the electrosurgery blade assembly includes a non-conductive tube member having a hollow tubular shaped opening and a slot where at least a portion of the conductive layer of the electrosurgery blade is positioned within the slot of the non-conductive tube member.

### CROSS-REFERENCE TO RELATED APPLICATIONS

This application is a continuation of, and claims priority to, Nonprovisional patent application having Ser. No. 15/695,642 filed Sep. 5, 2017, which claims priority to provisional patent application having Ser. No. 62/383,851 entitled “Monopolar Electrosurgery Blade and Electrosurgery Blade Assembly,” filed Sep. 6, 2016, which are herein incorporated by reference in their entireties.

### FIELD OF INVENTION

The present invention is generally directed to electrosurgery blades including electrosurgery blades having argon beam capability. More particularly, the present invention relates to a monopolar electrosurgery blade which includes a non-conductive planar member having opposite planar sides with a bottom angled sharp cutting edge, and a conductive layer located on one or both of the opposing planar sides of the non-conductive planar member where the conductive layer lies adjacent to the angled sharp cutting edge of the non-conductive planar member without covering the angled sharp cutting edge. In one exemplary embodiment of the electrosurgery blade, the conductive layer may form a closed loop shaped portion (and more particularly a closed generally triangular shaped loop portion) having an open interior through which a non-conductive opposing planar side is exposed. The non-conductive planar member may be tapered from a top of the non-conductive planar member to the bottom angled sharp cutting edge of the non-conductive planar member.

The present invention also relates to an electrosurgery blade assembly which includes the previously described monopolar electrosurgery blade plus a non-conductive tube member having a hollow tubular shaped opening, through which an inert gas can be supplied, and a slot which can be positioned over a portion of the electrosurgery blade. At least a portion of the conductive layer of the electrosurgery blade is positioned within the slot of the non-conductive tube member such that the hollow tubular shaped opening of the non-conductive tube member is positioned so that an inert gas supplied through the hollow tubular shaped opening will come in contact with at least a portion of the conductive layer of the electrosurgery blade thereby creating an ionized gas.

### BACKGROUND OF THE INVENTION

Typical electrosurgical pencils use an electrode blade which functions as an active electrode for use in performing cutting and coagulation during electrosurgery and a return electrode usually comprising an adhesive for attachment to a patient's skin. When the electrosurgery pencil is activated, the RF energy circulates from the active electrode to the return electrode through the patient's body with the distance between the active and return electrodes being fairly significant. Electrosurgery uses a RF generator and handpiece with an electrode to provide high frequency, alternating radio frequency (RF) current input at various voltages (2000-10,000V) depending on the function, namely coagulation vs. cutting. For cutting, heat generated from continuous RF high voltage conduction can create a vapor pocket which vaporizes and explodes a small section of tissue cells which results in an incision. Because of the heat generated, the lateral damage to the tissue is great and the possible necrosis of the tissue is high. For coagulation, voltage is usually lower than in cut mode and the slower heating process results in less heat. As a result, no vapor pocket is formed so the tissue for the most part remains intact but with cells and vessels destroyed and sealed at the point of contact.

It is also common to use argon beam coagulators during electrosurgery. In argon beam coagulation (ABC), plasma is applied to tissue by a directed beam of ionized argon gas (plasma) which causes a uniform and shallow coagulation surface thereby stopping blood loss. However, argon beam enhanced cutting may also be performed using application of an ionized argon gas.

At present, electrosurgery is often the best method for cutting and argon beam coagulation is often the best method for cessation of bleeding during surgery. Surgeons typically need to switch between argon beam coagulation and electrosurgery modes depending on what is happening during the surgery and what they need to achieve at a particular point in the surgery such as cutting, or making incisions in tissue, or stopping the bleeding at the surgical site.

However, since surgical tools and devices currently available to surgeons require switching between these two methods during the surgical procedure, there is a need for a surgical device or tool that enables a surgeon or user to utilize the best methods used for cutting and cessation of bleeding at the surgical site at the same time, or simultaneously, in addition to being able to use them separately. An electrosurgery blade having a sharp edge for cutting and RF and argon beam capability for capsulation would meet this need. The electrosurgery blades with a sharp edge and argon beam capability described with reference to the present invention could be used with an electrosurgery handpiece/pencil that does not have smoke evacuation capability but are also intended to be used with an electrosurgery handpiece/pencil that is capable of smoke evacuation during the electrosurgery procedure.

Such a surgical device or tool would enable the surgeon or user to increase both the efficiency and accuracy of the surgery by enabling the surgeon or user to perform both tissue cutting and coagulation at the same time without switching between modes or methods thereby decreasing operating time and reducing or eliminating the lateral damage to the tissue. In addition, performing both tissue cutting and coagulation at the same time along with smoke evacuation would protect the surgeon and staff form inhaling smoke and particles and also enable the surgeon or user to more clearly view the surgical site to ensure accuracy during the procedure without the need to stop and switch modes in order to stop bleeding at the surgery site before being able to clearly see the surgical site.

### SUMMARY OF THE INVENTION

The present invention is directed to an electrosurgery blade for use with an electrosurgery handpiece/pencil with smoke evacuation, or an electrosurgery handpiece/pencil without smoke evacuation, that includes a non-conductive planar member having opposite planar sides with opposing elongated edges and a sharp cutting edge, and a conductive layer located on one or both opposing planar sides where the conductive layer lies adjacent to the sharp cutting edge of the non-conductive planar member without covering the sharp cutting edge. The sharp cutting edge of the non-conductive layer is extremely sharp and capable of cutting biological tissue on its own without applying any power to the electrosurgery blade. The electrosurgery blade of the present invention is also extremely durable (won't break easily) and is resistant to high temperatures. The electrosurgery blade of the present invention is also capable of functioning at very low power levels (such as 15-20 watts) and up to three times lower power levels than existing electrosurgery blades that are used in electrosurgery pencils for cutting and coagulation.

In one exemplary embodiment, the conductive layer may form a closed loop shaped portion (and in particular a closed generally triangular shaped loop portion) having an open interior through which the non-conductive opposing planar side is exposed. The conductive layer may further comprise a rectangular shaped portion extending from the closed generally triangular shaped loop portion of the conductive layer.

The non-conductive planar member may comprise an inorganic, non-metallic solid material, such as a ceramic, for example. The conductive layer may comprise one or more materials such as, for example, stainless steel, copper, silver, gold, and/or titanium.

In another exemplary embodiment, there is a conductive layer that forms a closed loop shaped portion (and in particular a closed generally triangular shaped loop portion) located on each of the non-conductive opposite planar sides of the planar member where each of the closed loop shaped portions of the conductive layer (generally triangular in shape) extend to the opposing elongated edges of each respective opposite planar side and also each lie adjacent to the sharp cutting edge of the non-conductive planar member where the sharp cutting edge is a thin knife-like edge located at the bottom of the non-conductive planar member. The knife-like sharp cutting edge may be angled and the non-conductive planar member may be tapered from a top portion to the bottom portion to form the angled knife-like sharp cutting edge.

In yet another exemplary embodiment, the conductive layer covers a portion of the opposing elongated edges of each of the opposite planar sides such that it joins the closed loop portions (generally triangular in shape) located on each of the opposite planar sides by covering a top of the non-conductive planar member. In still another exemplary embodiment, the conductive layer may be present on only one of the non-conductive opposite planar sides such that it also extends over the top of the non-conductive planar member. In yet another exemplary embodiment, the electrosurgery blade may further comprise a shaft in communication with an end of a rectangular shaped portion of the conductive layer located opposite the closed loop portion(s) of the conductive layer where the shaft is conductive and is capable of being connected to an electrosurgery pencil. The sharp cutting edge of the non-conductive planar member is much thinner than the rest of the non-conductive planar member to enable precise cutting using the sharp cutting edge.

The present invention is also directed to an electrosurgery blade assembly which includes the previously described exemplary embodiments of the electrosurgery blade plus a non-conductive tube member having a hollow tubular shaped opening contained therein, through which an inert gas can be supplied, and a slot which can be positioned over a portion of the electrosurgery blade. At least a portion of the conductive layer of the electrosurgery blade is positioned within the slot of the non-conductive tube member such that the hollow tubular shaped opening of the non-conductive tube member is positioned so that an inert gas supplied through the hollow tubular shaped opening will come in contact with at least a portion of the conductive layer of the electrosurgery blade thereby creating an ionized gas. Like the non-conductive planar member, the non-conductive tube member may comprise an inorganic, non-metallic solid material, such as a ceramic, for example.

### BRIEF DESCRIPTION OF THE DRAWINGS

FIG. 1 is a top view of the non-conductive planar member of an exemplary embodiment of the monopolar electrosurgery blade of the present invention without the conductive layer;

FIG. 2 is a side view of the non-conductive planar member shown in FIG. 1;

FIG. 3 is a bottom view of the non-conductive planar member shown in FIGS. 1 and 2;

FIG. 4 is a side perspective view of an exemplary embodiment of the monopolar electrosurgery blade of the present invention;

FIG. 5 is a top view of the exemplary embodiment of the monopolar electrosurgery blade shown in FIG. 4;

FIG. 6 is an opposite side view of the exemplary embodiment of the monopolar electrosurgery blade shown in FIG. 4;

FIG. 7 is a bottom view of the exemplary embodiment of the monopolar electrosurgery blade shown in FIG. 4;

FIG. 8 is a schematic showing an exemplary embodiment of an electrosurgery blade assembly of the present invention which shows an exploded view of the positioning of a non-conductive tube member over the exemplary embodiment of the electrosurgery blade shown in FIG. 4 to provide the electrosurgery blade shown in FIG. 4 with argon beam capability;

FIG. 9 is a side perspective view of the exemplary embodiment of the electrosurgery blade assembly of the present invention depicted in FIG. 8; and

FIG. 10 is a magnified perspective view of the sharp cutting edge of the non-conductive planar member shown in FIG. 2.

### DETAILED DESCRIPTION OF EXEMPLARY EMBODIMENTS

The exemplary embodiments of the electrosurgery blade of the present invention enable a user or surgeon to use an electrosurgery blade having a non-conductive planar member with opposite planar sides and a sharp cutting edge, and a conductive layer located on one or both of the opposing sides, for cutting and/or coagulation. Exemplary embodiments of the electrosurgery blade assembly of the present invention include the exemplary embodiments of the electrosurgery blade of the present invention plus a non-conductive tube member having a hollow tubular shaped opening and a slot with at least a portion of the conductive layer of the electrosurgery blade positioned within the slot to enable a user or surgeon to separately use a sharp edged electrode for cutting and/or coagulation, separately use an argon beam for cutting and/or coagulation, or simultaneously use a sharp edged electrode and an argon beam for cutting and/or coagulation.

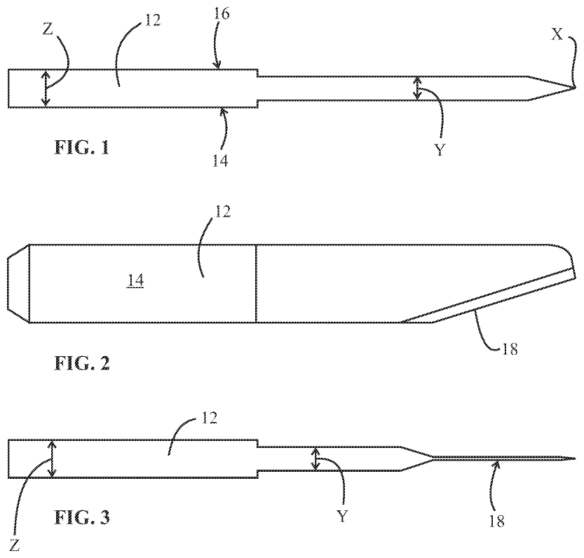

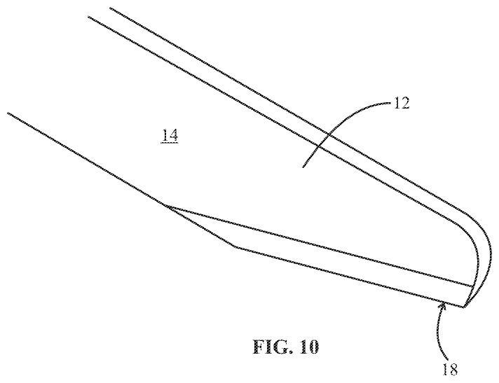

FIG. 1 shows a top view of the non-conductive planar member 12 of an exemplary embodiment of the monopolar electrosurgery blade of the present invention without the conductive layer. Non-conductive planar member 12 has opposite planar sides 14, 16. The top of non-conductive planar member 12 in FIG. 1 also shows non-conductive planar member 12 as having different widths along its length with the smallest width shown as a point X at the cutting end of the electrosurgery blade, a middle width Y, and a largest width Z shown at the non-cutting end of the electrosurgery blade where the blade is connected to an electrosurgery pencil. FIG. 2 is a side view of the non-conductive planar member 12 depicted in FIG. 1 which shows opposite planar side 14 and sharp cutting edge 18. Sharp cutting edge 18 is angled upward from a bottom elongated edge of opposite planar side 14. A magnified perspective view of sharp cutting edge 18 of the non-conductive planar member 12 is shown in FIG. 10. As can be seen in FIG. 10, non-conductive planar member 12 is tapered from a top portion to a bottom portion to create a non-conductive knife-like sharp cutting edge 18 at the bottom cutting end of the electrosurgery blade (the cutting end being the end of the electrosurgery blade opposite the end of the blade that is connected to an electrosurgery pencil). FIG. 3 is a bottom view of the non-conductive planar member 12 shown in FIGS. 1 and 2. FIG. 3 also shows the different widths of non-conductive planar member 12 and clearly shows sharp cutting edge 18 as having the smallest width given its knife-like sharp cutting edge.

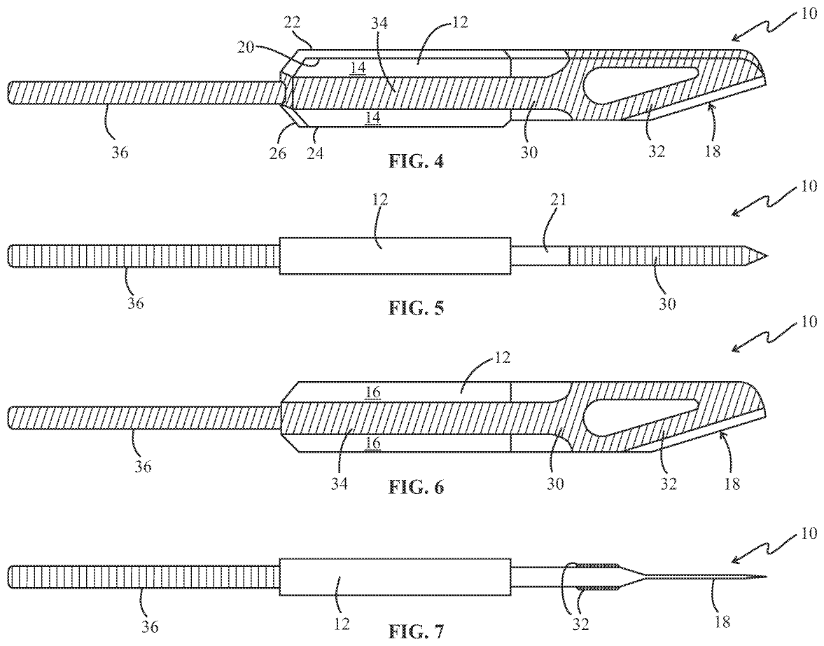

A side perspective view of an exemplary embodiment of the monopolar electrosurgery blade of the present invention is shown in FIG. 4. Monopolar electrosurgery blade 10 includes a non-conductive planar member 12 having opposite planar sides 14, 16 and a sharp cutting edge 18. Opposite planar sides 14, 16 have opposing elongated top edges 20, 22 and opposing elongated bottom edges 24, 26. Monopolar electrosurgery blade 10 also includes conductive layer 30. Conductive layer 30 has a generally triangular shaped closed loop portion 32 which is connected to a rectangular shaped portion 34. A conductive shaft 36 is connected to non-conductive planar member 12 opposite the sharp cutting edge 18 of non-conductive planar member 12. Rectangular shaped portion 34 of conductive layer 30 is connected to conductive shaft 36 by further extending conductive layer 30 so that it wraps around the non-cutting end of non-conductive planar member 12 so that it communicates with conductive shaft 36.

Although one exemplary embodiment of the monopolar electrosurgery blade of the present invention may have a conductive layer on only one opposite planar side of the non-conductive planar member, the exemplary embodiment of the monopolar electrosurgery blade 10 shown in FIGS. 4-7 has a conductive layer 30 contained on both opposite planar sides 14, 16 of the non-conductive planar member 12. The generally triangular shaped closed loop portions 32 of conductive layer 30 located on each of the opposite planar sides 14, 16 of the non-conductive planar member 12 are connected by extending the conductive layer 30 over the elongated top edges 20, 22 of the opposite planar sides 14, 16 and a top portion 21 of the non-conductive planar member 12. It will be understood by those skilled in the art that any number of configurations of conductive layer 30 may be used as long as a) the closed loop portions of the conductive layer have an opening therein and are located near the cutting end of the electrosurgery blade and above the non-conductive knife-like sharp cutting edge of the electrosurgery blade and b) the closed loop portions of the conductive layer are in communication with a conductive shaft that is attachable to an electrosurgery pencil.

The non-conductive planar member may comprise an inorganic, non-metallic solid material, such as a ceramic, for example. The conductive layer may comprise one or more materials such as, for example, stainless steel, copper, silver, gold, and/or titanium.

FIG. 5 is a top view of the exemplary embodiment of the monopolar electrosurgery blade 10 shown in FIG. 4. FIG. 5 shows the different widths of non-conductive planar member 12 as previously shown in FIG. 1 but also shows conductive layer 30 traversing part of top portion 21 of non-conductive planar member 12 near its cutting end and conductive shaft 36 attached to the non-cutting end of non-conductive planar member 12. FIG. 6 is an opposite side view of the exemplary embodiment of the monopolar electrosurgery blade shown in FIG. 4. Like opposite planar side 14 of non-conductive planar member 12, opposite planar side 16 of non-conductive planar member 12 has conductive layer 30 with a generally triangular shaped closed loop portion 32 which is connected to a rectangular shaped portion 34. Conductive shaft 36 is connected to non-conductive planar member 12 opposite the sharp cutting edge 18 of non-conductive planar member 12. Rectangular shaped portion 34 of conductive layer 30 is connected to conductive shaft 36 by further extending conductive layer 30 so that it wraps around the non-cutting end of non-conductive planar member 12 so that it communicates with conductive shaft 36. FIG. 7 is a bottom view of the exemplary embodiment of the monopolar electrosurgery blade shown in FIG. 4. FIG. 7 shows the different widths of non-conductive planar member 12 as previously shown in FIG. 3 but also shows generally triangular shaped closed loop portions 32 of conductive layer 30 located on opposite planar sides 14, 16 of non-conductive planar member 12 and conductive shaft 36 attached to the non-cutting end of non-conductive planar member 12. Unlike the top of monopolar electrosurgery blade 10 shown in FIG. 5, conductive layer 30 does not traverse a bottom portion of non-conductive planar member 12 near its cutting end to join generally triangular shaped closed loop portions 32.

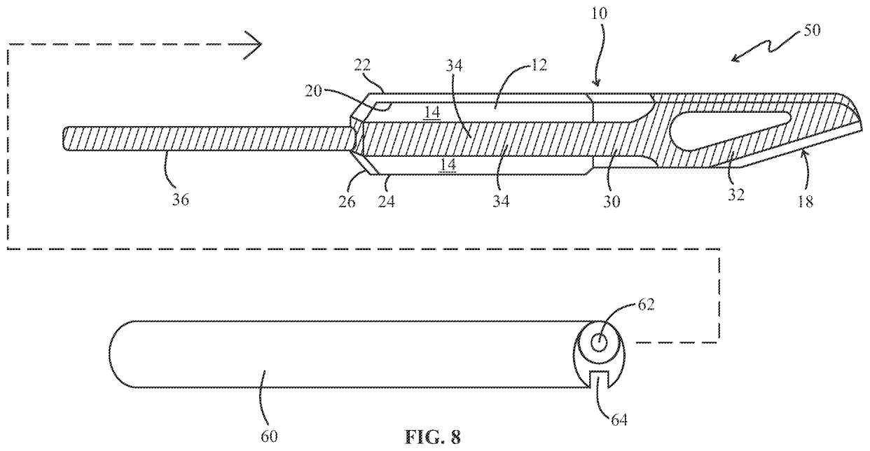

FIG. 8 is a schematic showing an exemplary embodiment of an electrosurgery blade assembly 50 of the present invention which shows an exploded view of the positioning of a non-conductive tube member 60 over the exemplary embodiment of the electrosurgery blade 10 shown in FIG. 4 to provide the electrosurgery blade shown in FIG. 4 with argon beam capability. Electrosurgery blade assembly 50 includes an electrosurgery blade 10 having a non-conductive planar member 12 with opposite planar sides 14, 16 and a sharp angled cutting edge 18 located on a bottom of the non-conductive planar member 12 where at least a portion of the non-conductive planar member 12 is tapered from a top of the non-conductive planar member 12 to the sharp angled cutting edge 18 on the bottom of the non-conductive planar member 12 (see also FIG. 10) and a conductive layer 30 located on at least one of the opposing planar sides 14, 16 of the non-conductive planar member 12 such that the conductive layer lies adjacent to the non-conductive sharp angled cutting edge 18. In this exemplary embodiment, a generally triangular shaped closed loop portions 32 of conductive layer 30 lies adjacent to the non-conductive sharp angled cutting edge 18. The electrosurgery blade assembly 50 also includes a non-conductive tube member 60 having a hollow tubular shaped opening 62 contained therein and a slot 64 contained therein where the slot 64 is positioned over at least a portion of the generally triangular shaped closed loop portions 32 of the conductive layer 30.

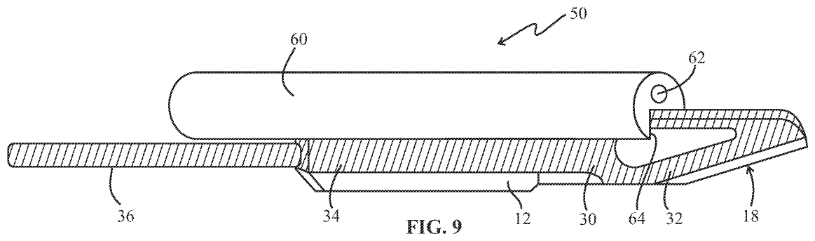

A side perspective view of the exemplary embodiment of the electrosurgery blade assembly 50 of the present invention depicted in FIG. 8 is shown in FIG. 9. The slot 64 of the non-conductive tube member 60 is positioned over at least a portion of the generally triangular shaped closed loop portions 32 of the conductive layer 30 and at least a portion of the non-conductive planar member 12. At least a portion of an outer surface of the non-conductive tube member 60 is located on each of the opposite planar sides 14, 16 of the non-conductive planar member 12. The hollow tubular shaped opening 62 of the non-conductive tube member 60 is positioned such that an inert gas supplied through the hollow tubular member shaped opening will come in contact with at least a portion of the generally triangular shaped closed loop portions 32 of the conductive layer 30. The non-conductive tube member may comprise an inorganic, non-metallic solid material, such as a ceramic, for example.

### Features and Advantages of the Electrosurgery Blade and Electrosurgery Blade Assembly of the Present Invention

The top of the non-conductive planar member is wider than the sharp cutting edge located on the bottom of the non-conductive planar member (as can be seen in FIGS. 3, 4 and 10).

The conductive layer located on one or both of the opposing sides of the non-conductive planar member may take on any number of configurations while still enabling the electrosurgery blade to function at very low power levels (such as 15-20 Watts or even less) while cutting and coagulating tissue.

The sharp non-conductive cutting edge of the electrosurgery blade can cut tissue without applying power to the electrosurgery blade and can also cut and coagulate tissue when power is applied to the electrosurgery blade.

The electrosurgery blade and electrosurgery blade assembly stop tissue from bleeding after cutting with minimal or no lateral damage to the tissue and without charring or burning of the tissue. Further, tissue does not stick to the electrosurgery blade or electrosurgery blade assembly while cutting and/or coagulating tissue. In addition, very little smoke is produced when using the electrosurgery blade or electrosurgery blade assembly due to the low or reduced power required for the electrosurgery blade to function.

The electrosurgery blade shown in FIGS. 4-7 can be used in any type of electrosurgery pencil that accommodates a monopolar electrode. The electrosurgery blade assembly shown in FIGS. 8 and 9 can be used in any type of electrosurgery pencil that accommodates a monopolar electrode and that is capable of providing an inert gas to the monopolar electrode.

The above exemplary embodiments are not intended to limit the scope, applicability, or configuration of the invention in any way. Rather, the disclosure is intended to teach both the implementation of the exemplary embodiments and modes and any equivalent modes or embodiments that are known or obvious to those reasonably skilled in the art. Additionally, all included figures are non-limiting illustrations of the exemplary embodiments and modes, which similarly avail themselves to any equivalent modes or embodiments that are known or obvious to those reasonably skilled in the art.

Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters, or other operating requirements without departing from the scope of the instant invention and are intended to be included in this disclosure.

1. An electrosurgery blade assembly comprising:
a non-conductive planar member having opposite planar sides and a sharp cutting edge;
a conductive layer located on at least one of the opposite planar sides of the non-conductive planar member such that the conductive layer lies adjacent to the sharp cutting edge without covering the sharp cutting edge; and
a non-conductive tube member having a hollow tubular shaped opening contained therein and a slot therein wherein the slot is positioned over at least a portion of the conductive layer.

2. The electrosurgery blade assembly of claim 1 wherein the conductive layer extends over a top of the non-conductive planar member.

3. The electrosurgery blade assembly of claim 1 wherein the sharp cutting edge is located on a bottom of the non-conductive planar member.

4. The electrosurgery blade assembly of claim 3 wherein the sharp cutting edge has a width that is less than half of a width of a top portion of the non-conductive planar member.

5. The electrosurgery blade assembly of claim 3 wherein at least a portion of the non-conductive planar member is tapered from a top of the non-conductive planar member to the bottom of the non-conductive planar member.

6. The electrosurgery blade assembly of claim 1 wherein a portion of the conductive layer forms a closed loop having an open interior through which the non-conductive opposite planar side is exposed.

7. The electrosurgery blade assembly of claim 1 wherein the slot of the non-conductive tube member is also positioned over at least a portion of the non-conductive planar member.

8. The electrosurgery blade assembly of claim 1 wherein at least a portion of an outer surface of the non-conductive tube member is located on each of the opposite planar sides of the non-conductive planar member.

9. The electrosurgery blade assembly of claim 8 wherein the hollow tubular shaped opening of the non-conductive tube member is positioned such that an inert gas supplied through the hollow tubular shaped opening will come in contact with at least a portion of the conductive layer.

10. An electrosurgery blade assembly comprising:
a non-conductive planar member having opposite planar sides, a top, a rounded end, and a sharp cutting edge located on a bottom of the non-conductive planar member wherein a portion of the sharp cutting edge lies beneath the rounded end of the non-conductive planar member;
a conductive layer located on at least one of the opposite planar sides of the non-conductive planar member such that the conductive layer extends over the rounded end and the top of the non-conductive planar member and lies adjacent to the sharp cutting edge without covering the sharp cutting edge; and
a non-conductive tube member having a hollow tubular shaped opening contained therein and a slot therein wherein the slot is positioned over at least a portion of the conductive layer.

11. The electrosurgery blade assembly of claim 10 further comprising another conductive layer located on the other opposite planar side of the non-conductive planar member.

12. The electrosurgery blade assembly of claim 11 further comprising a conductive layer located on a portion of the top of the non-conductive planar member that joins the conductive layers located on the opposite planar sides of the non-conductive planar member.

13. The electrosurgery blade assembly of claim 10 wherein the sharp cutting edge has a width that is less than half of a width of the top of the non-conductive planar member.

14. The electrosurgery blade assembly of claim 10 wherein at least a portion of the non-conductive planar member is tapered from the top of the non-conductive planar member to the bottom of the non-conductive planar member.

15. The electrosurgery blade assembly of claim 10 wherein a portion of the conductive layer forms a closed loop having an open interior through which the non-conductive opposite planar side is exposed.

16. The electrosurgery blade assembly of claim 10 further comprising a conductive shaft having opposing ends wherein one of the opposing ends is connected to an end of the non-conductive planar member located opposite the sharp cutting edge such that the conductive layer is in communication with the conductive shaft.

17. The electrosurgery blade assembly of claim 10 wherein said non-conductive planar member comprises a ceramic.

18. An electrosurgery blade assembly comprising:
a non-conductive planar member having opposite planar sides having opposing elongated top and bottom edges, an angled sharp cutting edge extending upward from the opposing elongated bottom edges, and a rounded end extending downward from the opposing elongated top edges;
a conductive layer located on each of the opposite planar sides such that the conductive layer lies adjacent to and covers a portion of the opposing elongated top edges of the opposite planar sides and the rounded end and at least a portion of a top of the non-conductive planar member such that it joins the conductive layer covering the portions of the opposing elongated top edges of the opposing planar sides; and
a non-conductive tube member having a hollow tubular shaped opening contained therein and a slot therein wherein the slot is positioned over at least a portion of the conductive layer.

19. The electrosurgery blade assembly of claim 18 wherein the conductive layer forms a first closed generally triangular shaped loop portion having an open interior through which one of the non-conductive opposite planar sides is exposed wherein the first closed generally triangular shaped loop portion of the conductive layer lies adjacent to the angled sharp cutting edge of the non-conductive planar member without covering the non-conductive angled sharp cutting edge.

20. The electrosurgery blade assembly of claim 19 wherein the conductive layer further comprises a first rectangular shaped portion extending from the first closed generally triangular shaped loop portion and a conductive shaft having opposing ends wherein one of the opposing ends is in communication with an end of the first conductive rectangular shaped portion wherein the conductive shaft is capable of being connected to an electrosurgery pencil.

21. The electrosurgery blade assembly of claim 20 wherein the conductive layer further forms a second closed generally triangular shaped loop portion having an open interior through which the other non-conductive opposite planar side is exposed wherein the second closed generally triangular shaped loop portion of the conductive layer lies adjacent to the angled sharp cutting edge of the non-conductive planar member without covering the non-conductive angled sharp cutting edge.

22. The electrosurgery blade assembly of claim 21 wherein the conductive layer further comprises a second rectangular shaped portion extending from the second closed generally triangular shaped loop portion wherein the second rectangular shaped portion is in communication with an end of the conductive shaft.
